SEQUENCE LISTING (12 sequences)

SEQ ID NO: 1 - DNA, 21 bp, synthetic construct
  source 1..21: mol_type = other DNA
tgcgtgtggagtatttggatg

SEQ ID NO: 2 - DNA, 22 bp, synthetic construct
  source 1..22: mol_type = other DNA
tggtacagtcagagccaacctc

SEQ ID NO: 3 - DNA, 20 bp, synthetic construct
  source 1..20: mol_type = other DNA
gtggtagtgcaagctgaagg

SEQ ID NO: 4 - DNA, 20 bp, synthetic construct
  source 1..20: mol_type = other DNA
ttcagccagttgcccatcta

SEQ ID NO: 5 - DNA, 20 bp, synthetic construct
  source 1..20: mol_type = other DNA
cagcctcaagatcatcagca

SEQ ID NO: 6 - DNA, 20 bp, synthetic construct
  source 1..20: mol_type = other DNA
tgtggtcatgagtccttcca

SEQ ID NO: 7 - DNA, 37 bp, synthetic construct
  source 1..37: mol_type = other DNA
tatcgataggtaccggaaagcaatgaacaaataaatc

SEQ ID NO: 8 - DNA, 33 bp, synthetic construct
  source 1..33: mol_type = other DNA
gatcgcagatctcgaccggagcccagcagctac

SEQ ID NO: 9 - DNA, 37 bp, synthetic construct
  source 1..37: mol_type = other DNA
catagaagattctagatgaagtcgatcctagatggcc

SEQ ID NO: 10 - DNA, 34 bp, synthetic construct
  source 1..34: mol_type = other DNA
agatccttcgcggcctcacagagcctcggcagac

SEQ ID NO: 11 - DNA, 35 bp, synthetic construct
  source 1..35: mol_type = other DNA
catagaagattctagatggaggaatgctgggtgac

SEQ ID NO: 12 - DNA, 36 bp, synthetic construct
  source 1..36: mol_type = other DNA
agatccttcgcggcctcagcaatcagagaggtctag

(19) United States
(12) Patent Application Publication
(10) Pub. No.: US 2026/0335340 A1
(43) Pub. Date: Oct. 29, 2026

(54) PHARMACEUTICAL COMPOSITION FOR TREATING LIVER CANCER COMPRISING CANNABIDIOL AND ANTICANCER AGENT AS ACTIVE INGREDIENTS AND USE THEREOF

(71) Applicants: NEOCANNBIO CO., LTD., Seoul (KR); KOREA INSTITUTE OF SCIENCE AND TECHNOLOGY, Seoul (KR)

(72) Inventors: Jeong Kook KIM, Seoul (KR); Jung Ho SONG, Seoul (KR); Jungyeob HAM, Gangneung-si (KR); Young-Joo KIM, Gangneung-si (KR); Taejung KIM, Gangneung-si (KR); Youngsic JEON, Gangneung-si (KR)

(73) Assignees: NEOCANNBIO CO., LTD., Seoul (KR); KOREA INSTITUTE OF SCIENCE AND TECHNOLOGY, Seoul (KR)

(21) Appl. No.: 19/111,283

(22) PCT Filed: Sep. 13, 2023
(86) PCT No.: PCT/KR2023/013739

§ 371 Date: Mar. 12, 2025

(30) Foreign Application Priority Data
Sep. 14, 2022 (KR) 10-2022-0115644

(51) Int. Cl. A61K 31/00; A61K 31/47; A61P 35/00

(52) U.S. Cl. CPC A61K 31/658; A61K 31/47; A61P 35/00

(57) ABSTRACT

The present disclosure relates to a pharmaceutical composition for preventing or treating liver cancer, including cannabidiol (CBD) and an anticancer agent as active ingredients, and use thereof. It was confirmed that the composition of the present disclosure exhibits remarkably excellent liver cancer inhibition efficacy when cannabidiol and cabozantinib, which is one of the liver cancer anticancer agents, are used in combination, and therefore, liver cancer treatment effects may be enhanced by using the composition.

### TECHNICAL FIELD

The present disclosure relates to a pharmaceutical composition for preventing or treating liver cancer including cannabidiol (CBD) and an anticancer agent as active ingredients, and use thereof.

### BACKGROUND ART

Cannabis (Cannabis sativa L.) is an annual plant of the genus Cannabis in the family Cannabaceae, which has been widely cultivated in tropical and temperate regions, centered around Central Asia, for over 12,000 years, including wild hemp, and Cannabis chemovars and their variants, including variants var. indica and var. kafiristanica, which contain various kinds of cannabinoid compounds known as medicinal and pharmaceutical ingredients, and Cannabis sativa subspecies sativa, Cannabis sativa subspecies indica, Cannabis sativa subspecies ruderalis, and genetically crossbred, self-bred, or hybrid plants thereof.

In traditional Chinese and Korean medical texts, hulled cannabis seed, called majain or hwamain, has been used for constipation, diabetes, pain disorders, irregular menstruation, skin diseases, and dysentery, and cannabis weed, which is cannabis leaf (mayeop), has been used as an anthelmintic, hair protector, asthma, analgesic, anesthetic, diuretic, etc. Additionally, cannabis root has been used to treat difficult labor and relieve blood stasis, cannabis skin has been used to treat bruises and open wounds, cannabis flower has been used to treat paralysis and itching, etc., and cannabis stem fiber has been used to treat difficult labor, constipation, gout, insanity, insomnia, etc., and there are records of using each part of cannabis according to the corresponding symptom.

Cannabis includes about 400 compounds, most of which are cannabinoids, terpenes, and phenolic compounds, and of these, there are about 90 cannabinoids, which are medicinally and pharmaceutically important natural ingredients, many of which are ingredients found only in cannabis. Among the cannabinoids of cannabis, a psychoactive component is Δ9-tetrahydrocannabinol (Δ9-THC), and cannabidiol (CBD) is a non-psychoactive component known to exhibit physiologically active effects through various receptors in the human body, including adrenergic receptors and cannabinoid receptors.

Meanwhile, according to an analysis by Statistics Korea, cancer is the number one cause of death in the Republic of Korea, accounting for 31.7% of men and 22.5% of women, and 31.7% of the entire population dies of cancer. There are methods of treating cancer, such as surgical treatment, radiation therapy, and treatment through administration of anticancer agents and the like, but these treatment methods are accompanied by side effects or are applied in a limited manner depending on the degree of cancer progression. In particular, mechanisms of most anticancer agents function by stopping the cell cycle of cells that are actively dividing and causing them to die, so they attack cancer cells as well as cells that are normally dividing, resulting in side effects such as hair loss, loss of appetite, and decreased immunity due to a decrease in white blood cells. Therefore, a technology is needed to maximize the therapeutic effect while using a low concentration of anticancer agents.

Under these circumstances, the present disclosure was completed by confirming significantly improved anticancer efficacy through combined treatment of anticancer agents and cannabidiol.

### DISCLOSURE OF INVENTION

### Technical Problem

One aspect provides a pharmaceutical composition for preventing or treating liver cancer, including cannabidiol (CBD) and an anticancer agent as an active ingredient.

Another aspect provides a pharmaceutical composition for enhancing an anticancer effect of liver cancer, including cannabidiol (CBD).

### Solution to Problem

One aspect is to provide a pharmaceutical composition for preventing or treating liver cancer, including cannabidiol (CBD) and an anticancer agent as an active ingredient.

As used herein, the term “Cannabidiol (CBD)” is one of the main compounds constituting cannabis represented by the following Formula 1 (IUPAC name: 2-[(1R,6R)-3-methyl-6-prop-1-en-2-ylcyclohex-2-en-1-yl]-5-pentylbenzene-1,3-diol)), and is a substance that is often compared to tetrahydrocannabinol (THC), which causes hallucinations. In Korea, cannabidiol is designated as a narcotic and not much research is being conducted, but in reality, has almost no psychotropic effect, so in foreign countries, cannabidiol is used for medical purposes to alleviate symptoms such as pain, memory impairment, and anxiety, etc., and is known to suppress excessive oil production, so research related to cosmetics ingredients for acne skin is also actively being conducted. Additionally, there are several reports suggesting that cannabidiol may be effective in epileptic syndromes that cause seizures by participating in the excitatory-inhibitory equilibrium of neural activity.

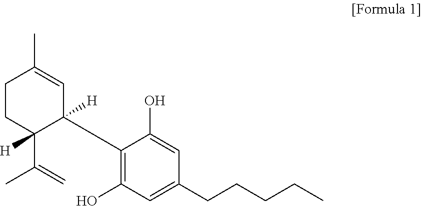 [SMILES: C=C(C)[C@@H]1CCC(C)=C[C@H]1Cc1c(O)cc(CCCCC)cc1O]

The cannabidiol may include one or more selected from the group consisting of derivatives of cannabidiol, stereoisomers thereof, pharmaceutically acceptable salts thereof, hydrates thereof, and solvates thereof.

As used herein, the term “pharmaceutically acceptable salt” refers to a form of a salt that may be used pharmaceutically among salts, which are substances in which cations and anions are bonded by electrostatic attraction, and may typically be a metal salt, a salt with an organic base, a salt with an inorganic acid, a salt with an organic acid, a salt with a basic or acidic amino acid, etc. For example, the metal salts may be alkali metal salts (sodium salt, potassium salt, etc.), alkaline earth metal salts (calcium salt, magnesium salt, barium salt, etc.), aluminum salts, etc.; the salts with organic bases may be salts with triethylamine, pyridine, picoline, 2,6-lutidine, ethanolamine, diethanolamine, triethanolamine, cyclohexylamine, dicyclohexylamine, N,N-dibenzylethylenediamine, etc.; the salts with inorganic acids may be salts with hydrochloric acid, hydrobromic acid, nitric acid, sulfuric acid, phosphoric acid, etc.; the salts with organic acids may be salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, etc., salts with basic amino acids may be salts with arginine, lysine, ornithine, etc.; salts with acidic amino acids may be salts with aspartic acid, glutamic acid, etc. Particularly preferable salts are, when the compound has an acidic functional group therein, inorganic salts such as alkali metal salts (for example, sodium salts, potassium salts, etc.), alkaline earth metal salts (for example, calcium salts, magnesium salts, barium salts, etc.), etc., and organic salts such as ammonium salts; and, when the compound has a basic functional group therein, salts with inorganic acids such as hydrochloric acid, hydrobromic acid, nitric acid, sulfuric acid, phosphoric acid, etc., and salts with organic acids such as acetic acid, phthalic acid, fumaric acid, oxalic acid, tartaric acid, maleic acid, citric acid, succinic acid, methanesulfonic acid, p-toluenesulfonic acid, etc.

As used herein, the term “cancer” refers to a tumor that has grown abnormally due to the autonomous excessive growth of body tissue, or a disease that forms a tumor. The cancer may be a solid cancer, and specifically, may be liver cancer. Furthermore, the ‘liver cancer’ commonly refers to hepatocellular carcinoma (HCC), and refers to a malignant tumor that has developed from hepatocytes that make up the liver. Primary liver cancer accounts for about 90% of liver cancers. Liver cancer is likely to develop in patients with underlying liver disease, and is known that 75-85% of liver cancer patients have cirrhosis, and 10-30% of cirrhosis patients develop liver cancer.

In the composition, the cannabidiol and the anticancer agent may be co-administered, and specifically, the cannabidiol may be administered simultaneously with the anticancer agent in one formulation, or simultaneously or sequentially with the anticancer agent in a separate formulation.

As used herein, the term “co-administration” may be achieved by administering individual ingredients of a treatment regimen simultaneously, sequentially, or individually. Co-administration is a method of obtaining a combination treatment effect by administering two or more drugs simultaneously or sequentially, or alternately at regular or indefinite intervals, etc., concomitant therapy is not limited thereto, but may be defined as a method in which the efficacy measured through the degree of response, the rate of response, the period until disease progression, or the survival period is therapeutically superior to the efficacy that may be obtained by administering one or the remaining ingredients of the concomitant therapy at a typical dose and may provide a synergistic effect.

The composition may include the cannabidiol and the anticancer agent in a concentration ratio of 9:1 to 1:9. Specifically, the concentration ratio of cannabidiol and anticancer agent (cannabidiol:anticancer agent) may be 9:1 to 1:9, 9:1 to 1:7, 9:1 to 1:5, 9:1 to 1:4, 9:1 to 1:3, 9:1 to 1:2, 9:1 to 1:1, 7:1 to 1:9, 7:1 to 1:7, 7:1 to 1:5, 7:1 to 1:4, 7:1 to 1:3, 7:1 to 1:2, 7:1 to 1:1, 5:1 to 1:9, 5:1 to 1:7, 5:1 to 1:5, 5:1 to 1:4, 5:1 to 1:3, 5:1 to 1:2, 5:1 to 1:1, 4:1 to 1:9, 4:1 to 1:7, 4:1 to 1:5, 4:1 to 1:4, 4:1 to 1:3, 4:1 to 1:2, 4:1 to 1:1, 3:1 to 1:9, 3:1 to 1:7, 3:1 to 1:5, 3:1 to 1:4, 3:1 to 1:3, 3:1 to 1:2, 3:1 to 1:1, 2:1 to 1:9, 2:1 to 1:7, 2:1 to 1:5, 2:1 to 1:4, 2:1 to 1:3, 2:1 to 1:2, 2:1 to 1:1, 1:1 to 1:9, 1:1 to 1:7, 1:1 to 1:5, 1:1 to 1:4, 1:1 to 1:3, or 1:1 to 1:2.

The cannabidiol may be included in the composition at a concentration of 1 to 50 μM. Specifically, the cannabidiol may be included at a concentration of 1 to 50 μM, 1 to 45 μM, 1 to 40 μM, 1 to 35 μM, 1 to 30 μM, 1 to 25 μM, 1 to 20 μM, 1 to 15 μM, 5 to 50 μM, 5 to 45 μM, 5 to 40 μM, 5 to 35 μM, 5 to 30 μM, 5 to 25 μM, 5 to 20 μM, 5 to 15 μM, 10 to 50 μM, 10 to 45 μM, 10 to 40 μM, 10 to 35 μM, 10 to 30 μM, 10 to 25 μM, 10 to 20 μM, 10 to 15 μM, 15 to 50 μM, 15 to 45 μM, 15 to 40 μM, 15 to 35 μM, 15 to 30 μM, 15 to 25 μM, 15 to 20 μM, 20 to 50 μM, 20 to 45 μM, 20 to 40 μM, 20 to 35 μM, 20 to 30 μM or 20 to 25 μM.

Additionally, the cannabidiol may be included in the composition in a concentration range that does not exhibit toxicity, inhibition and/or apoptosis effects on liver cancer cells or liver cancer tissue.

The anticancer agent may be included in the composition at a concentration of 1 to 100 μM. Specifically, the anticancer agent may be included at a concentration of 1 to 100 μM, 1 to 80 μM, 1 to 60 μM, 1 to 50 μM, 1 to 45 μM, 1 to 40 μM, 1 to 35 μM, 1 to 30 μM, 1 to 25 μM, 1 to 20 μM, 1 to 15 μM, 5 to 100 μM, 5 to 80 μM, 5 to 60 μM, 5 to 50 μM, 5 to 45 μM, 5 to 40 μM, 5 to 35 μM, 5 to 30 μM, 5 to 25 μM, 5 to 20 μM, 5 to 15 μM, 10 to 100 μM, 10 to 80 μM, 10 to 60 μM, 10 to 50 μM, 10 to 45 μM, 10 to 40 μM, 10 to 35 μM, 10 to 30 μM, 10 to 25 μM, 10 to 20 μM, 10 to 15 μM, 15 to 100 μM, 15 to 80 μM, 15 to 60 μM, 15 to 50 μM, 15 to 45 μM, 15 to 40 μM, 15 to 35 μM, 15 to 30 μM, 15 to 25 μM, 15 to 20 μM, 20 to 100 μM, 20 to 80 μM, 20 to 60 μM, 20 to 50 μM, 20 to 45 μM, 20 to 40 μM, 20 to 35 μM, 20 to 30 μM or 20 to 25 μM. The anticancer agent may be an anticancer agent for liver cancer, and may include one or more selected from the group consisting of Cabozantinib, Sorafenib, Lenvatinib, and Regorafenib, and may specifically include Cabozantinib.

As used herein, the term “Cabozantinib” is a compound represented by the following Formula 2, which is an anticancer agent used to treat medullary thyroid cancer, renal cell carcinoma, and hepatocellular carcinoma, and is known to inhibit tyrosine kinase c-Met and VEGFR2.

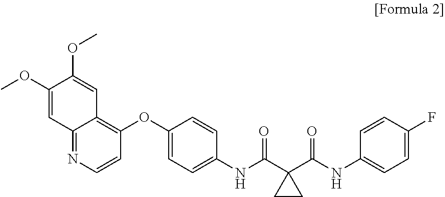 [SMILES: COc1cc2nccc(Oc3ccc(NC(=O)C4(C(=O)Nc5ccc(F)cc5)CC4)cc3)c2cc1OC]

The cabozantinib may include one or more selected from the group consisting of derivatives of cabozantinib, stereoisomers thereof, pharmaceutically acceptable salts thereof, hydrates thereof, and solvates thereof.

As used herein, the term “treatment” refers to any action that improves or beneficially changes the symptoms of liver cancer by administering the composition of the present disclosure.

As used herein, the term “prevention” refers to any action by which the possibility of developing liver cancer or a disease is suppressed or delayed by administration of the composition of the present disclosure.

The composition may more effectively induce inhibition of proliferation and apoptosis of liver cancer cells by co-administering cannabidiol and an anticancer agent. Specifically, the composition may induce or promote one or more of the effects of apoptosis and autophagy of liver cancer cells.

According to an embodiment, the composition was confirmed to induce an increase in the expression of apoptosis-related factors, PARP, Cleaved PARP, Bax, Cleaved caspase-3, Cleaved caspase-9, and Cleaved caspase-8 proteins, and may be seen that an anticancer effect was significantly enhanced when cannabidiol and an anticancer agent were administered together.

According to an embodiment, the composition was confirmed to increase phosphorylation levels of Chk2, c-Jun and/or p53, and it may be seen that the anticancer effect was significantly enhanced when cannabidiol and an anticancer agent were administered together.

According to an embodiment, the composition was confirmed to induce increased expression of GRP78 protein, an ER stress-related factor, and PERK phosphorylation, and it may be seen that the anticancer effect was significantly enhanced when cannabidiol and an anticancer agent were administered together.

In an embodiment, the CBD or a composition including the CBD may enhance the anticancer effect of an anticancer agent against liver cancer, and specifically, may enhance the anticancer effect of cabozantinib against liver cancer. More specifically, the CBD may improve and/or enhance the cancer treatment effect of an anticancer agent by inducing or enhancing the activity of the anticancer agent, reducing the resistance of cancer cells to the anticancer agent, or increasing the sensitivity/sensitivity to the anticancer agent.

The pharmaceutical composition may include a pharmaceutically acceptable carrier. The “pharmaceutically acceptable carrier” may refer to a carrier or diluent that does not stimulate an organism and does not inhibit the biological activity and properties of a compound being injected. Here, “pharmaceutically accepted” means that it does not inhibit the activity of the active ingredient and does not have any toxicity beyond what the subject of application (prescription) may adapt to. Any type of carrier that may be used in the pharmaceutical composition may be used as long as the carrier is a pharmaceutically accepted carrier commonly used in the art. Non-limiting examples of the carrier may be lactose, dextrose, maltodextrin, sucrose, sorbitol, mannitol, xylitol, erythritol, maltitol, glycerol, ethanol, starch, acacia gum, alginate, gelatin, calcium phosphate, calcium silicate, cellulose, methyl cellulose, microcrystalline cellulose, polyvinyl pyrrolidone, water, saline, sterile water, Ringer's solution, buffered saline, albumin injection solution, methylhydroxybenzoate, propylhydroxybenzoate, talc, magnesium stearate, or mineral oil, etc. They may be used alone or in combination of two or more. The pharmaceutical composition may be prepared as an oral formulation or a parenteral formulation depending on an administration route by a typical method known in the art, including a pharmaceutically accepted carrier in addition to the active ingredient.

The pharmaceutical composition may be formulated and used in the form of an oral formulation such as a pill, granule, tablet, capsule, suspension, emulsion, syrup, aerosol, etc., topical preparation, suppository, or sterile injectable solution, each according to typical methods.

The pharmaceutical composition may be formulated and used in the form of an oral formulation such as a pill, granule, tablet, capsule, suspension, emulsion, syrup, aerosol, etc., topical preparation, suppository, or sterile injectable solution, each according to typical methods. When formulating the pharmaceutical composition, the pharmaceutical composition may be prepared by adding diluents or excipients such as commonly used filler, bulking agent, binder, humectant, disintegrant, or surfactant, etc.

When the pharmaceutical composition is prepared as an oral formulation, the pharmaceutical composition may be prepared in the form of a powder, granule, tablet, pill, dragee, capsule, liquid, gel, syrup, suspension, wafer, etc., along with a suitable carrier, according to methods known in the art. Examples of pharmaceutically accepted suitable carriers may be sugars such as lactose, glucose, sucrose, dextrose, sorbitol, mannitol, and xylitol, etc., starches such as corn starch, potato starch, and wheat starch, etc., celluloses such as cellulose, methylcellulose, ethylcellulose, sodium carboxymethylcellulose, and hydroxypropylmethylcellulose, etc., polyvinyl pyrrolidone, water, methylhydroxybenzoate, propylhydroxybenzoate, magnesium stearate, mineral oil, malt, gelatin, talc, polyols, and vegetable oils. When formulating, the formulation may include diluents and/or excipients such as a filler, bulking agent, binder, humectant, disintegrant, and surfactant, etc. as needed.

When the pharmaceutical composition is prepared as a parenteral formulation, the pharmaceutical composition may be formulated into the form of an injection, a transdermal administration agent, a nasal inhalant, and suppository along with a suitable carrier according to methods known in the art. When formulated as an injectable formulation, suitable carriers may include sterile water, ethanol, polyols such as glycerol or propylene glycol, or mixtures thereof, desirably Ringer's solution, phosphate buffered saline (PBS) containing triethanolamine, sterile water for injection, and isotonic solutions such as 5% dextrose, etc. may be used. When formulated as a transdermal agent, the transdermal agent may be formulated in the form of an ointment, cream, lotion, gel, topical solution, paste, liniment, and aerosol, etc. For a nasal inhalant, the nasal inhalant may be formulated in the form of an aerosol spray using a suitable propellant such as dichlorofluoromethane, trichlorofluoromethane, dichlorotetrafluoroethane, carbon dioxide, etc., when formulated as a suppository, the base used may be witepsol, tween 61, polyethylene glycol, cacao butter, laurin butter, polyoxyethylene sorbitan fatty acid esters, polyoxyethylene stearate, sorbitan fatty acid esters, etc.

The pharmaceutical composition may be administered in a pharmaceutically effective amount, wherein the term “pharmaceutically effective amount” means an amount sufficient to treat or prevent a disease at a reasonable benefit/risk ratio applicable to medical treatment or prevention, and the effective dosage level may be determined depending on the severity of the disease, the activity of the drug, the patient's age, weight, health, sex, the patient's sensitivity to the drug, the time of administration, the route of administration and the excretion rate of the composition of the present disclosure used, the treatment period, the drug used in combination with or concurrently with the composition of the present disclosure used, and other factors well known in the medical field. The pharmaceutical composition may be administered alone or in combination with an ingredient known to exhibit a therapeutic effect on a known liver cancer disease. Taking all of the factors into account, it is important to administer the minimum amount that may achieve the maximum effect without side effects.

The dosage of the pharmaceutical composition may be determined by a person skilled in the art considering the purpose of use, the degree of addiction of the disease, the patient's age, weight, gender, pre-existing condition, or the type of substance used as an active ingredient, etc. For example, the pharmaceutical composition of the present disclosure may be administered at a dose of from about 0.1 ng to about 1,000 mg/kg per adult, desirably from 1 ng to about 100 mg/kg, and the frequency of administration of the compositions of the present disclosure is not specifically limited, but may be administered once daily or administered multiple times in divided doses. The dosage or frequency of administration does not limit the scope of the present disclosure in any way.

Another aspect is to provide a pharmaceutical composition for enhancing the anticancer effect of liver cancer, including cannabidiol (CBD). The same parts as described above also apply to the composition.

The composition may be a composition for co-administration with an anticancer agent, and specifically, the composition may be administered in combination with an anticancer agent.

The cannabidiol may be included in the composition in a concentration range that does not exhibit toxicity, inhibition and/or apoptosis effects on liver cancer cells or liver cancer tissue.

As used herein, the term “enhancing cancer treatment effect” or “enhancing anticancer effect” may refer to improving and/or enhancing the cancer treatment effect of an anticancer agent by inducing or enhancing the activity of the anticancer agent, reducing the resistance of cancer cells to the anticancer agent, or increasing the receptivity/sensitivity to the anticancer agent.

In an embodiment, the CBD or a composition including the CBD may enhance the anticancer effect of an anticancer agent against liver cancer, and specifically, may enhance the anticancer effect of cabozantinib against liver cancer.

Another aspect provides a method of treating or preventing liver cancer, which includes administering to a subject a pharmaceutical composition for preventing or treating liver cancer or a pharmaceutical composition for enhancing an anticancer effect of liver cancer. The same aspects as described above apply equally to the method.

As used herein, the term “subject” may include, without limitation, mammals, birds, reptiles, farmed fish, etc., including mice, livestock, humans, etc., that develop or are at risk of developing liver cancer, and the subject may exclude humans.

The pharmaceutical composition may be administered in single or multiple doses in a pharmaceutically effective amount. At this time, the composition may be formulated and administered in the form of a liquid, powder, aerosol, injection, infusion (Ringel), capsule, pill, tablet, suppository, or patch. The administration route of the pharmaceutical composition for preventing or treating liver cancer may be by any common route as long as the pharmaceutical composition may reach the target tissue.

The pharmaceutical composition may be administered by routes such as, but is not particularly limited to, intraperitoneal administration, intravenous administration, intramuscular administration, subcutaneous administration, intradermal administration, transdermal patch administration, oral administration, intranasal administration, intrapulmonary administration, or rectal administration, etc., depending on the intended purpose. However, when administered by oral administration, the pharmaceutical composition may be administered in an unformulated form, and since the active ingredient of the pharmaceutical composition may be denatured or decomposed by gastric acid, the oral composition may be administered orally in a form that coats the active agent or is formulated to protect the active agent from decomposition in the stomach, or in the form of an oral patch. Additionally, the composition may be administered by any device that may transport the active substance to target cells.

Another aspect provides a method of enhancing treating or the anticancer effect of liver cancer, which includes administering to a subject a pharmaceutical composition for preventing or treating liver cancer or a pharmaceutical composition for enhancing the anticancer effect of liver cancer. The same aspects as described above apply equally to the method.

The method may enhance the anticancer effect of an anticancer agent against liver cancer by administering the CBD or a composition including the CBD to a subject, and specifically, may enhance the anticancer effect of cabozantinib against liver cancer.

The method may further include administering an anticancer agent to the subject. The method may include co-administering an anticancer agent and cannabidiol.

Another aspect provides a use of a composition including cannabidiol (CBD) and an anticancer agent as active ingredients for the prevention or treatment of liver cancer. The same parts as described above also apply to the use.

Another aspect provides a use of cannabidiol (CBD) or a composition including cannabidiol as an active ingredient for treating or enhancing the anticancer effect of liver cancer. The same parts as described above also apply to the use.

### Advantageous Effects of Invention

It was confirmed that the composition of the present disclosure exhibits remarkably excellent liver cancer inhibition efficacy when cannabidiol and cabozantinib, one of the liver cancer anticancer agents, are used in combination, therefore, the liver cancer treatment effect may be enhanced by using the composition.

### BRIEF DESCRIPTION OF DRAWINGS

FIG. 1 is a diagram illustrating a cannabidiol preparation process.

FIG. 2 is a diagram illustrating survival rates of HepG2 cells according to treatment with different concentrations of cannabidiol.

FIG. 3 is a diagram illustrating results of Western blotting analysis of expression levels of cleaved PARP, cleaved caspase9, cleaved caspase8, and Bax in HepG2 cells treated with different concentrations of cannabidiol.

In FIG. 4, A is a diagram illustrating the survival rates of HepG2 cells according to combined treatment with cannabidiol and cabozantinib, and B is a diagram illustrating the results of Western blotting analysis of the expression levels of cleaved PARP, cleaved caspase9, cleaved caspase8, cleaved caspase3, Bax, and Bcl-2.

In FIG. 5, A and B are diagrams confirming the results of apoptosis according to combined treatment with cannabidiol and cabozantinib.

In FIG. 6, A and B are diagrams confirming protein activation levels of related signal transduction pathways according to combined treatment with cannabidiol and cabozantinib.

FIG. 7, A and B are diagrams confirming the apoptotic effects and protein activation levels of related signaling pathways according to treatment with BML-277 after combined treatment with cannabidiol and cabozantinib.

In FIG. 8, A and B are diagrams confirming cell viability and apoptotic effects according to TP53 knockdown when treated with a combination of cannabidiol and cabozantinib.

FIG. 9 is a diagram confirming a significant correlation between the expression levels of genes related to p53 activation and ER stress-related genes through analysis of a public transcriptome data set.

In FIG. 10, A is a diagram confirming expression levels of GRP78 and TP53 according to combined treatment with cannabidiol and cabozantinib, B is a diagram confirming a promoter region and protein expression levels of TP53, and C is a diagram confirming expression levels of phosphorylated PERK and eIF2a.

In FIG. 11, A is a diagram confirming cell viability and GRP78 levels according to LAA treatment when treated with a combination of cannabidiol and cabozantinib, B is a diagram confirming activation levels of P53 and Chk2, and C is a diagram confirming expression levels of cleaved PARP and cleaved caspase8.

In FIG. 12, A and B are diagrams confirming cell viability according to CNR1 or CNR2 expression when treated with cannabidiol and cabozantinib in combination.

In FIG. 13, A is a diagram confirming results of apoptosis according to CNR1 or CNR2 expression when treated with cannabidiol and cabozantinib in combination, and B is a diagram confirming expression levels of cleaved PARP, cleaved caspase8, and cleaved caspase9.

(*p<0.05, **p<0.01, and ***p<0.001; Student's t-test).

### MODE FOR THE INVENTION

The present disclosure will be explained in more detail in the following embodiments. However, these embodiments are for illustrative purposes only and the scope of the disclosure is not limited to these embodiments.

### Example 1: Cannabis Origin and Information

The green hemp (cannabis, Cannabis sativa L.) used in the present disclosure was cultivated in a licensed facility within the Gangneung Research Institute of the Korea Institute of Science and Technology (KIST) (Gangneung-si, Gangwon-do). The cultivation and use of green hemp was carried out in compliance with regulations and guidelines regarding cannabis research. Furthermore, the cannabidiol compounds used in the examples below were manufactured and used based on the Seoul Regional Food and Drug Safety Office's Narcotics (Cannabis) Academic Researcher's Permit (No. 1564, No. 1979, and No. 2083).

### Example 2: Cannabidiol (CBD) Purification

Cannabis leaves were harvested in July 2020, dried, and then finely chopped.

Naturally dried leaves (500 g) were extracted twice with ethanol (5 L) at room temperature to obtain an ethanol extract (37 g). Next, the extract was suspended in water and separated with normal hexane to obtain a residue (16 g).

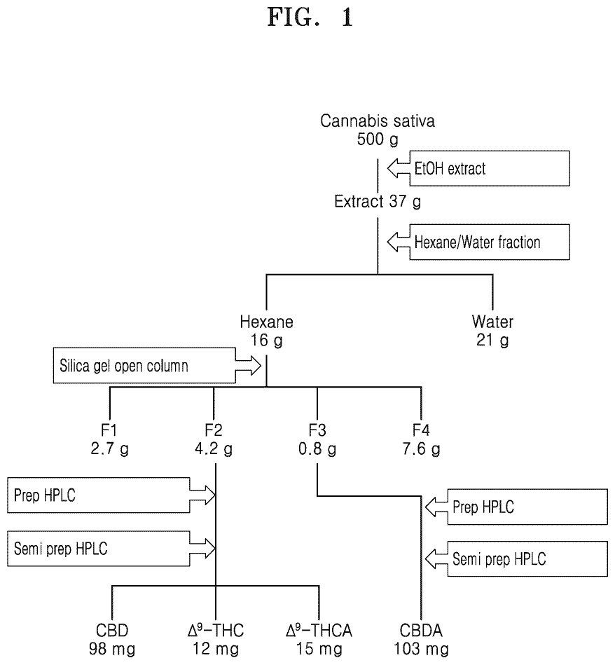

Subsequently, silica open column chromatography (Merck, Darmstadt, Germany, 230-400 mesh, 7.5×10.0 cm ID) was conducted stepwise using hexane:ethyl acetate eluent. The F2 fraction (4.2 g) acquired above was applied to preparative HPLC (Phenomenex Luna C18 column; 250×21.2 mm, 10 m) eluted with water (A) and MeCN (B) at a flow rate of 10 mL/min using a 220 nm UV detector through a gradient solvent system (70 to 85% MeCN, 60 min), acquiring four subfractions. Each subfraction was further purified by semi-preparative HPLC (Phenomenex Luna C18 (2); 250×10 mm, 5 m) eluting with 70-85% MeCN at a flow rate of 4 mL/min to acquire pure substances including CBD (98 mg), D9-THC (12 mg), and D9-THCA (15 mg). Additionally, the F3 fraction (0.8 g) was subjected to a similar purification process as above to acquire cannabidiolic acid (CBDA) (103 mg). Specifically, the process is disclosed in FIG. 1.

### Example 3: Cell Culture Method and Compound Acquisition

Human HCC cell lines (HepG2 and Hep3B) were purchased from the American Type Culture Collection (ATCC). The cell lines were cultured in MEM (Gibco, Carlsbad, MD, USA) and DMEM (Gibco) supplemented with 10% FBS (Gibco), 100 U/mL penicillin, and 100 g/mL streptomycin, respectively.

Cabozantinib, a compound used in the present disclosure, was purchased from Sigma-Aldrich (St. Louis, MO, USA), and BML-277 was purchased from Selleck Chemicals (Houston, TX, USA).

### Example 4: Cell Viability Analysis

Cells (1.0×10⁴) were seeded in 96-well plates and cultured in a medium for 24 hours. The cultured cells were treated with various concentrations of CBD and/or cabozantinib, etc., for 48 hours, and then cell viability was measured using a WST-8 assay kit (Biomax, SEOUL, Korea).

### Example 5: Western Blotting Analysis

Proteins (whole cell extract, 30 g/lane) from protein samples were separated by electrophoresis on NuPAGE 4-12% Bis-Tris gels (Invitrogen, Carlsbad, CA, USA), blotted onto PVDF transfer membranes, and analyzed using epitope-specific primary and secondary antibodies. The conjugated antibodies were visualized using the SuperSignal™ WestFemto Maximum sensitivity substrate (Thermo Fisher Scientific, San Jose, CA, USA) and the LAS 4000 imaging system (Fujifilm, Japan).

### Example 6: Fluorescence-Activated Cell Sorting (FACS) Analysis

Apoptotic cells were analyzed by flow cytometry using the FITC Annexin Apoptosis Detection Kit (BD Biosciences, CA, USA). HepG2 cells were divided into four groups (for example, control group, CBD, CBZ, and combination treatment groups). After treatment with the compounds for 48 hours, FITC Annexin V and propidium iodide were added to the washed cells and cultured at room temperature for 15 minutes. Apoptotic cells were measured using a FACSVerse Flow Cytometer (BD Biosciences), and data were analyzed using Flowjo software (version 10.8.1, BD, Ashland, OR, USA). At least 1000 cells were counted for data analysis.

### Example 7: Multi Phospho-Kinase Assay

Multi phospho-kinase activities were measured using the Human Phospho-Kinase array system (R&D System, Minneapolis, NM, USA) according to the manufacturer's recommendations.

### Example 8: Real-Time PCR Analysis

Quantitative real-time PCR was conducted using iQ™ SYBR Supermix (Bio-Rad, Hercules, CA, USA) to amplify mRNA of GRP78 and TP53. Primer sequences and thermal cycling conditions are listed in the table below.

TABLE 1

| | Annealing | Nucleic acid | SEQ |
|---|---|---|---|
| Primer | conditions | sequence (5′-3′) | ID NO: |
| TP53 F | 58° C. | TGCGTGTGGAGTATTT | 1 |
| | | GGATG | |
| TP53 R | | TGGTACAGTCAGAGCC | 2 |
| | | AACCTC | |
| GRP78 F | 58° C. | GTGGTAGTGCAAGCTG | 3 |
| | | AAGG | |
| GRP78 R | | TTCAGCCAGTTGCCCA | 4 |
| | | TCTA | |
| GAPDH F | 58° C. | CAGCCTCAAGATCATC | 5 |
| | | AGCA | |
| GAPDH R | | TGTGGTCATGAGTCCT | 6 |
| | | TCCA | |

### Example 9: Dual-Luciferase Reporter Assay

TP53 promoter activity was assessed using a dual-luciferase reporter assay. Specifically, to determine the effect on TP53 promoter activity, cells were co-transfected with a reporter construct including pGL3.0-basic (Promega, Madison, WI, USA) and TP53-luciferase included in a pNL1.1.TK vector. At 24 hours after transfection, luciferase activity was measured using the Nano-Glo® Dual Luciferase® Reporter Assay System (Promega) according to the manufacturer's protocol. Relative firefly luciferase activity was normalized to NanoLuc™ luciferase activity to account for variations in transfection efficiency.

### Example 10: Transcriptome Data Analysis

For transcriptome data analysis, publicly available data from The Cancer Genome Atlas-Liver Hepatocellular Carcinoma (TCGA-LIHC) data set were utilized, obtained using the TCGAbiolinks package in R software. Furthermore, data were acquired from the GSE4024 (accessed on 12 Jul. 2023), GSE89377 (accessed on 12 Jul. 2023), and GSE6764 (accessed on 12 Jul. 2023) datasets of the NCBI GEO database (https://www.ncbi.nlm.nih.gov/geo/, accessed on 12 Jul. 2023). To ensure consistency, samples including normal tissue were excluded from the analysis.

Correlation plots were generated using R software, and gene ontology information was retrieved from the gene ontology database (http://www.geneontology.org/, accessed on 12 Jul. 2023) and the reactome database (http://www.reactome.org/, accessed on 12 Jul. 2023).

### Example 11: Cloning and Establishment of Overexpression Cell Lines

Total RNA was extracted from normal liver tissues and cDNA was synthesized to clone a tagged CNR1 and CNR2 coding gene sequences into a pCDH-CMV-EF1-puro vector. The CNR1 and CNR2 genes were amplified by PCR using specific primers including 5′-extension and NotI (NEB, Ipswich, MA, USA) and XbaI (NEB) restriction sites. CloneAmp HiFi PCR Premix (Thermo Fisher Scientific, San Jose, CA, USA) was used for PCR reactions. Subsequently, amplicons were obtained from the PCR products by gel extraction, digested with NotI and XbaI enzymes, and cloned into the pCDH-CMV-EF1-puro vector using the In-Fusion® cloning system according to the manufacturer's instructions. Primer sequences and thermal cycling conditions are listed in the table below.

TABLE 2

| | | | SEQ |
|---|---|---|---|
| | Annealing | Nucleic acid | ID |
| Primer | conditions | sequence (5′-3′) | NO: |
| TP53 promoter − | 65 °C. | TATCGATAGGTACCGG | 7 |
| 500 bp F | | AAAGCAATGAACAAAT | |
| | | AAATC | |
| TP53 promoter + | | GATCGCAGATCTCGAC | 8 |
| 50 bp R | | CGGAGCCCAGCAGCTA | |
| | | C | |
| CNR1 | 65 °C. | CATAGAAGATTCTAGA | 9 |
| infusion F | | TGAAGTCGATCCTAGA | |
| | | TGGCC | |
| CNR1 | | AGATCCTTCGCGGCCT | 10 |
| infusion R | | CACAGAGCCTCGGCAG | |
| | | AC | |
| CNR2 | 65 °C. | CATAGAAGATTCTAGA | 11 |
| infusion F | | TGGAGGAATGCTGGGT | |
| | | GAC | |
| CNR2 | | AGATCCTTCGCGGCCT | 12 |
| infusion R | | CAGCAATCAGAGAGGT | |
| | | CTAG | |

HepG2 cells were transfected with pCDH-CMV-EF1-puro including the tagged CNR1 and CNR2 coding sequences together with gag-pol and VSV-G plasmids (plasmids 14887 and 8454, respectively; Addgene, Cambridge, MA, USA). Transfection was conducted using Lipofectamine® 3000 (Invitrogen, Carlsbad, CA, USA) according to the manufacturer's instructions. After transfection, cells were treated with puromycin (0.5-1.0 g/mL, Sigma Aldrich Co., St. Louis, MO, USA) for 4 weeks to select stable cell lines expressing CNR1 or CNR2. To confirm the specificity and efficiency of CNR1 and CNR2 overexpression, mRNA expression levels were analyzed using RT-PCR.

### Example 12: SiRNA-Mediated Knockdown Experiment

p53 (siRNA; sc-29435, Santa Cruz, CA, USA), c-Jun (siRNA; sc-29223, Santa Cruz), or MISSION@siRNA Universal Negative Control (Sigma-Aldrich Co.) were transfected into HepG2 cells using Lipofectamine® RNAiMAX Transfection Reagent (Invitrogen). To confirm the specificity and efficiency of p53 and c-Jun inhibition, protein expression levels were analyzed using Western blotting.

### Example 13: Statistical Analysis

The experimental results are expressed as a mean±standard deviation from at least three independent results for each experiment. Statistical significance was analyzed using Student's t-test. All p-values are two-tailed, and p-values less than 0.05 were considered significant.

### Experimental Example 1: Evaluation of Cannabidiol's Cancer Cell Toxicity

To evaluate the toxicity of cannabidiol (CBD) on liver cancer cells, the following experiments were conducted.

Specifically, HepG2 cells or Hep3B cells, which are hepatocellular carcinoma (HCC) cell lines, were seeded at a concentration of 1×10⁴ cells/well in a 96-well plate and cultured for 24 hours in an incubator at 37° C. and 10% CO₂, then, cannabidiol was treated at various concentrations and cultured under the same conditions for 48 hours. After completion of the culture, 1/10 of the WST-8 solution (Quanti-Max™, BIOMAX, Korea) was added to the medium, and the culture was further conducted at 37° C. for 1 hour, the absorbance was measured at 450 nm to determine the cell viability, which was expressed as a ratio to the untreated control group. This experiment was conducted independently and repeated three times.

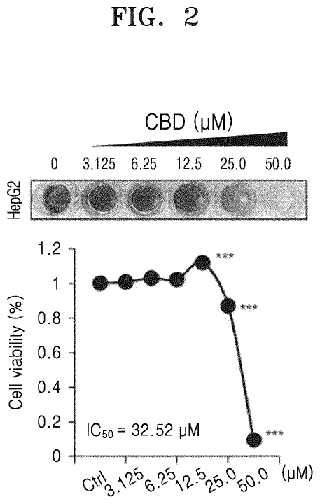

As a result, the IC₅₀ value for cannabidiol in HepG2 cells was confirmed to be 32.52 μM (FIG. 2), indicating that cannabidiol does not exhibit toxicity to liver cancer cells at concentrations below this level.

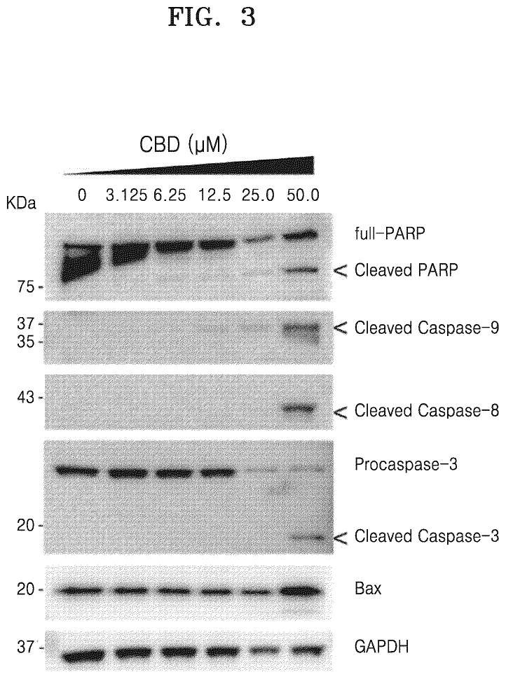

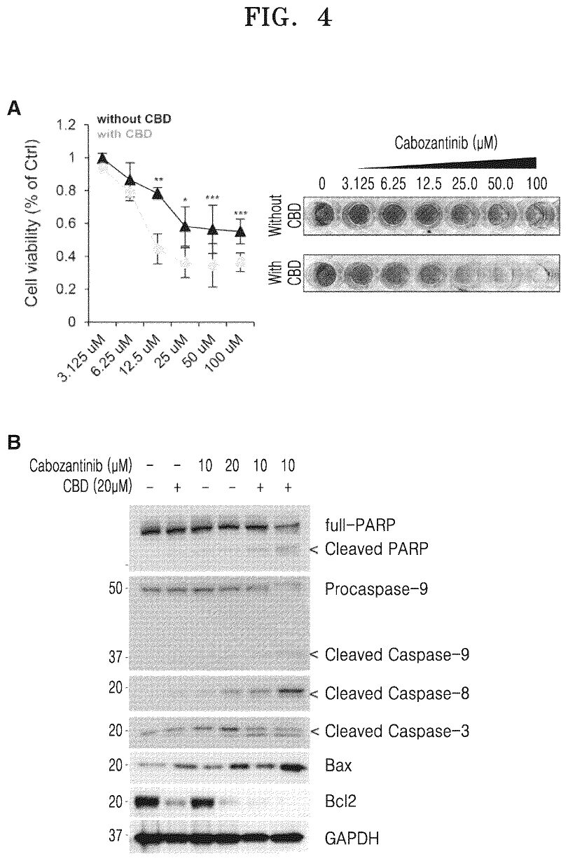

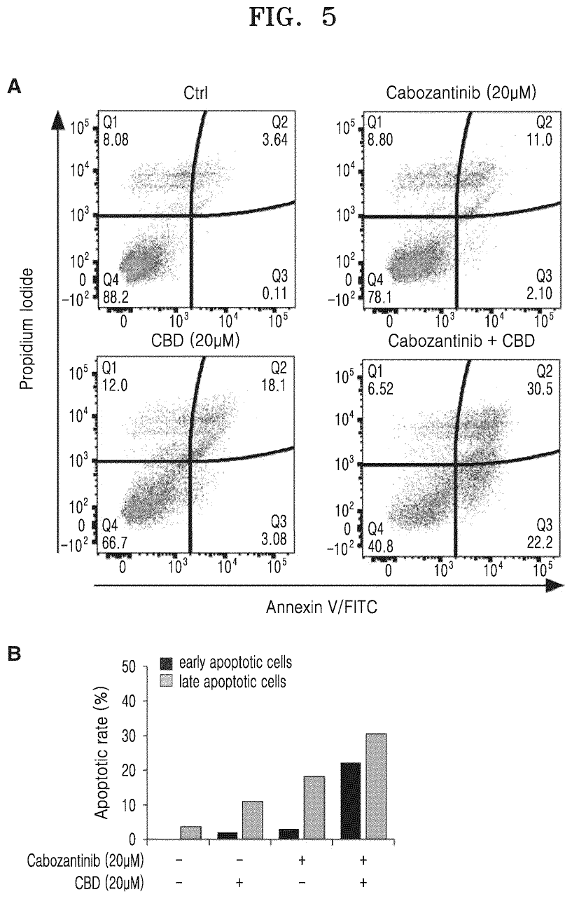

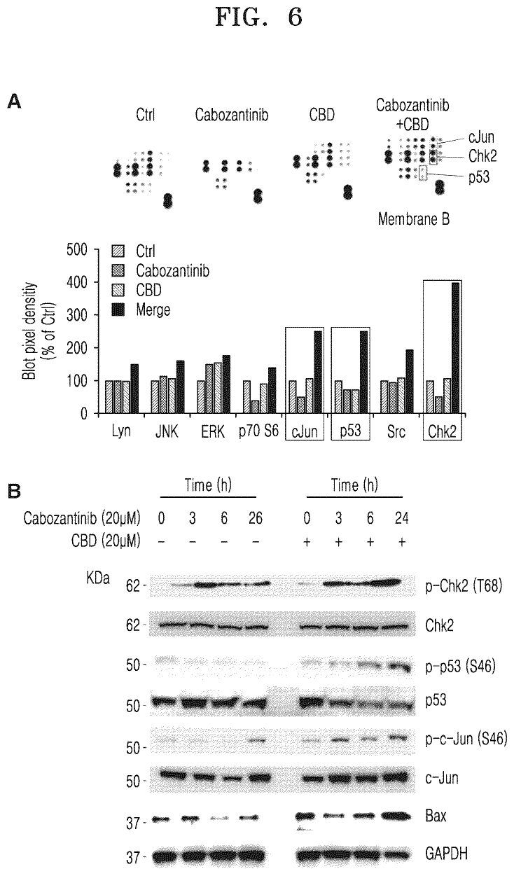

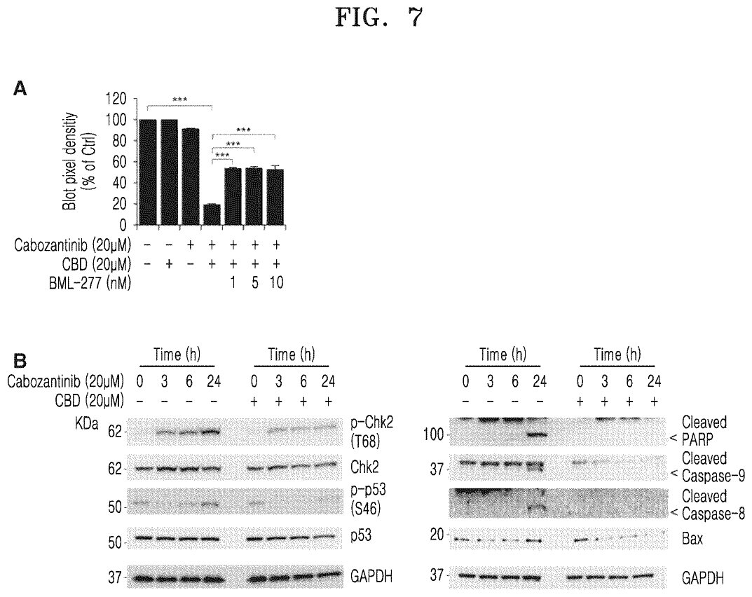

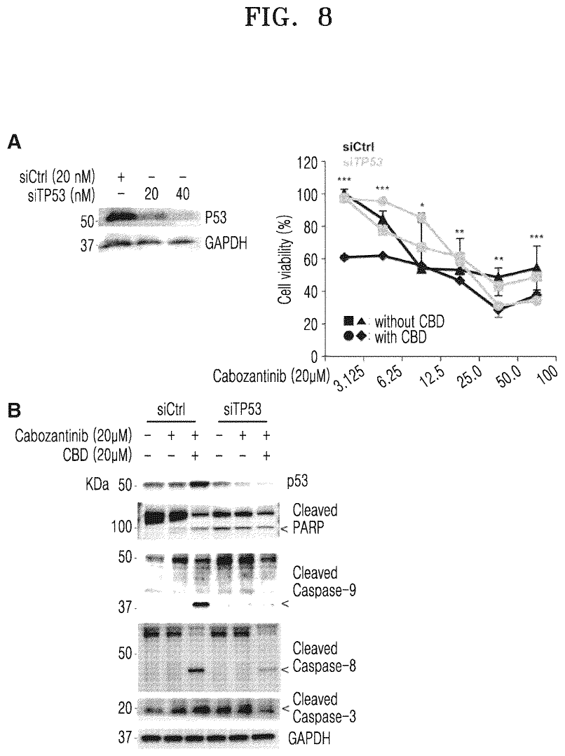

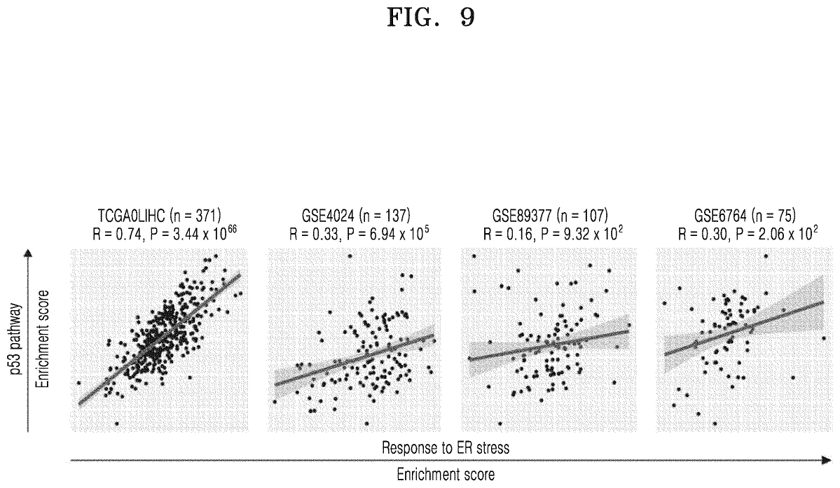

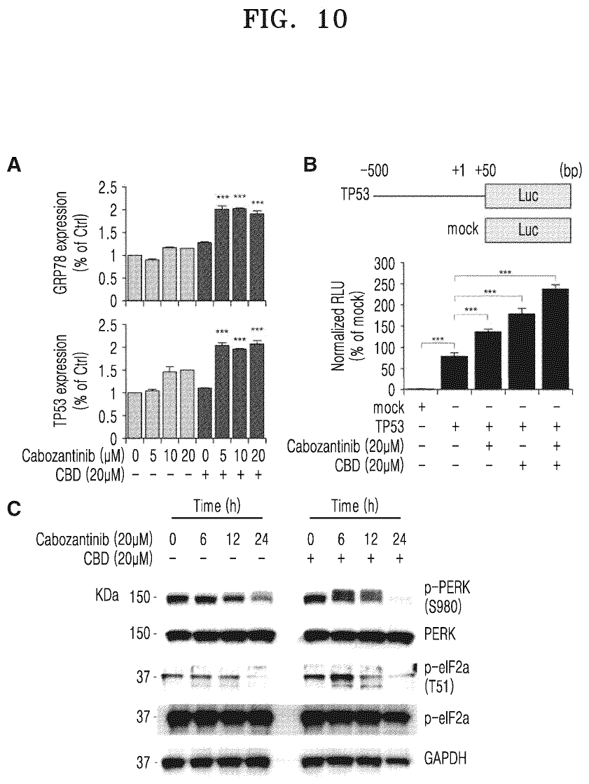

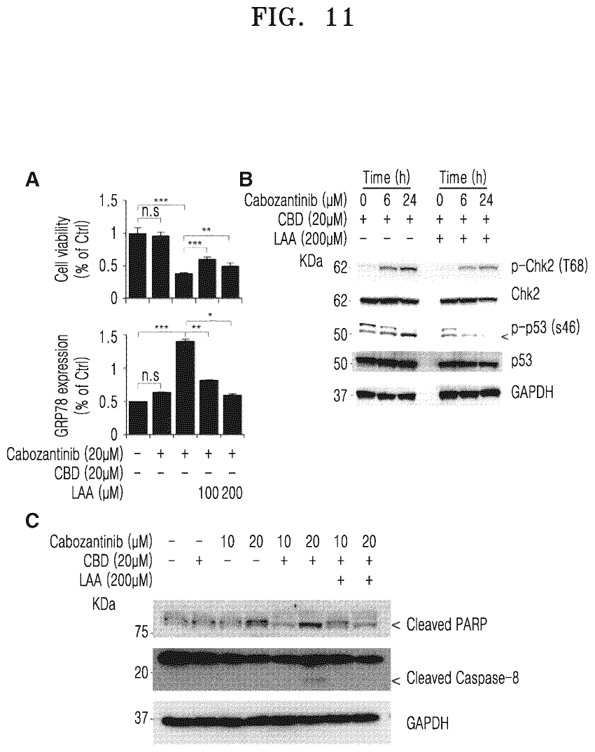

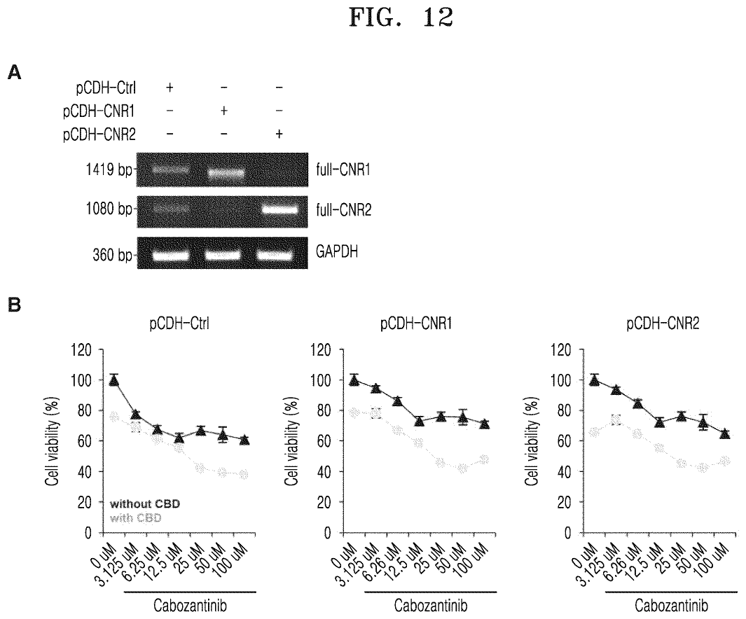

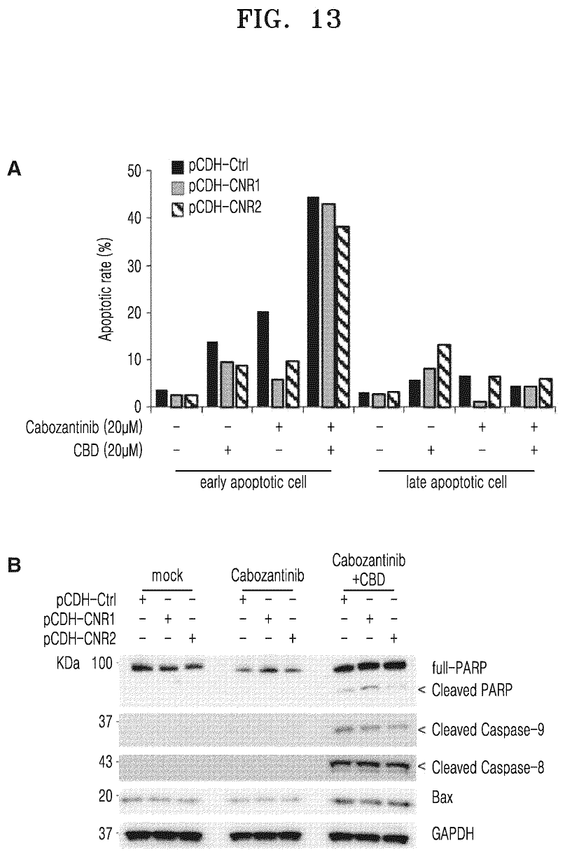

Subsequently, the expression levels of apoptotic marker proteins such as Bax (Bcl2-associated X) and the cleavage levels of caspase-9, caspase-8, caspase-3, and PARP (poly (ADP-ribose) polymerase) were analyzed to further confirm the apoptotic effect of cannabidiol at various concentrations (0, 3.125, 6.25, 12.5, 25.0, and 50.0 μM). As a result, it was confirmed that cell death induced by cannabidiol was concentration-dependent (FIG. 3). In particular, no significant effect was observed at relatively low concentrations of cannabidiol (<25 μM).

Therefore, based on the experimental results, subsequent experiments were conducted with the cannabidiol concentration set to 20 μM.

### Experimental Example 2: Evaluation of Cancer Cell Killing Efficacy of Combined Treatment with Cannabidiol and Cabozantinib

To evaluate the cancer cell killing efficacy of cabozantinib, known as a liver cancer treatment, when combine treated with cannabidiol (CBD), the following experiments were conducted.

Specifically, HepG2 cells, a hepatoma cell line, were seeded at a concentration of 1×10⁴ cells/well in a 96-well plate and cultured for 24 hours in an incubator at 37° C. and 10% CO₂, then they were treated with cabozantinib at various concentrations and treated or not with cannabidiol (20 μM), and then cultured under the same conditions for 48 hours. After completion of the culture, 1/10 of the WST-8 solution (Quanti-Max™, BIOMAX, Korea) was added to the medium, and the culture was further conducted at 37° C. for 1 hour, the absorbance was measured at 450 nm to determine the cell viability, which was expressed as a ratio to the untreated control group. This experiment was conducted independently and repeated three times. Additionally, as positive control groups, sorafenib, lenvatinib, or regorafenib were treated instead of cabozantinib, respectively.

As a result, it was confirmed that the toxicity of cabozantinib against HepG2 cells was significantly increased when cannabidiol and cabozantinib were combine treated compared to cabozantinib treatment alone (IC₅₀=15.76 μM when combine treated with 20 μM CBD), and it was confirmed that cannabidiol significantly increases the anticancer effect of cabozantinib among various anticancer agents (Table 3 and FIG. 4A).

TABLE 3

IC₅₀ (μM), 48 h

| Drug | without CBD | with CBD |
|---|---|---|
| Sorafenib | 14.58 | 9.24 |
| Cabozantinib | >100 | 15.76 |
| Lenvatinib | >100 | >100 |
| Regorafenib | 14.07 | 9.74 |

Subsequently, the combined treatment effect of cabozantinib with other cannabis-related compounds, such as cannabidiolic acid (CBDA), tetrahydrocannabinol (THC), and THCA, in addition to cannabidiol were examined, and as a result, it was confirmed that cannabidiol showed a significantly superior combined treatment effect with cabozantinib among the various cannabis compounds (Table 4).

TABLE 4

| | IC₅₀ (μM), 48 h |
|---|---|
| Drug | with Cabozantinib |
| CBDA | 35.85 ± 1.35 |
| CBD | 15.76 ± 0.75 |
| THC | 23.9 ± 0.55 |
| THCA | 30.35 ± 0.85 |

Subsequently, it was evaluated whether the combined treatment of cabozantinib and cannabidiol may induce apoptosis. As a result, it was confirmed that, the levels of various apoptosis protein markers were significantly increased when cabozantinib and cannabidiol were combine treated compared to CBD or cabozantinib treatment alone, while conversely, the expression of Bcl-2, an apoptosis stimulation inhibitor, showed a pattern of decreasing (FIG. 4B).

Next, the apoptosis rate was analyzed using FACS, and it was confirmed that the ratio of early and late apoptotic cells significantly increased when cabozantinib and cannabidiol were combine treated, compared to CBD or cabozantinib treatment alone (FIG. 5).

Based on the results, it may be seen that combined treatment with cabozantinib and cannabidiol may significantly increase the anticancer effect against cancer, which indicates that cannabidiol enhances the sensitivity of cancer cells (especially liver cancer cells) to apoptosis induced by cabozantinib.

### Experimental Example 3: Mechanism of Action Analysis of Combined Treatment with Cannabidiol and Cabozantinib

To determine the mechanism by which combination treatment with cabozantinib and CBD, multiple phosphokinase assays were conducted to identify signal transduction pathways modulated by combined treatment.

First, kinase proteins suspected of being involved in cell proliferation, differentiation, cell cycle regulation, and apoptosis using a multi-phospho-kinase assay were screened. As a result, it was confirmed that, that the levels of phosphorylated Chk2 (checkpoint kinase 2), phosphorylated c-Jun, and phosphorylated p53 were significantly increased when combine treated compared to single treatment with cabozantinib or CBD (FIG. 6A). Proteins with increased phosphorylation levels are associated with apoptosis, and Chk2 activation induces apoptosis and cell cycle arrest through p53 phosphorylation. In addition, the phosphorylation levels of Chk2, c-Jun, and p53 were examined over time when treated with cabozantinib alone and combine treated with cabozantinib and CBD, and it was confirmed that they were significantly increased when combine treated (FIG. 6B).

Next, to determine whether Chk2 activated by combined treatment with cabozantinib and CBD affects apoptosis, the anti-apoptotic effect mediated by Chk2 inhibitors was confirmed. Treatment with BML-277, an ATP-competitive inhibitor of Chk2, inhibited the apoptotic effect of combined treatment with cabozantinib and CBD (FIG. 7A). In addition, the effect of BML-277 treatment on the levels of phosphorylated Chk2 and p53 and apoptotic proteins in combined treatment with cabozantinib and CBD was examined, and as a result, it was confirmed that BML-277-mediated Chk2 inhibition reduced the phosphorylation of p53 and the levels of apoptotic proteins (FIG. 7B).

Next, to confirm the p53-related mechanism by combined treatment with cabozantinib and CBD, the level of apoptosis according to the level of p53 expression was evaluated. Specifically, HepG2 cells in which the p53 gene was knocked down (transfected with siTP53) using a small interfering RNA (siRNA) system were treated with CBD and cabozantinib in a dose-dependent manner, resulting in increased cell survival compared to cells transfected with siCtrl (siCtrl vs. siTP53, p<0.01; FIG. 8A). Furthermore, the level of apoptotic proteins by combined treatment with cabozantinib and CBD in HepG2 cells in which the p53 gene was knocked down was examined, as a result, it was confirmed that cells transfected with siTP53 showed a significant decrease in the level of the apoptotic proteins (FIG. 8B).

### Experimental Example 4: Analysis of ER Stress and TP53 Activation Following Combined Treatment with Cannabidiol and Cabozantinib

To confirm the association between P53 activation and endoplasmic reticulum (ER) stress by combined treatment with cabozantinib and CBD, the following experiments were conducted.

First, human transcriptome data sets including TCGA-LIHC, GSE4024, GSE89377, and GSE6764, were analyzed. The analysis results confirmed a significant correlation between the expression levels of p53 activation-related genes and ER stress-related genes in independent cohorts such as TCGA-LIHC, GSE4024, and GSE6764 (FIG. 9).

In addition, the expression level of GRP78 (glucose-regulated protein 78), an ER stress marker induced by combined treatment with cabozantinib and CBD was investigated. As a result, it was confirmed that the expression levels of GRP78 and TP53 increased when combine treated with cabozantinib or CBD compared to single treatment (FIGS. 10A and B).

Next, to monitor cellular ER stress induced by combined treatment with cabozantinib and CBD, the levels of phosphorylated PERK (protein kinase RNA-like endoplasmic reticulum kinase) and eIF2a (eukaryotic initiation factor 2 alpha) was evaluated, and it was confirmed that combined treatment increased the levels of phosphorylated proteins compared to single treatment (FIG. 10C).

Next, the effect of L-ascorbic acid (LAA), known to alleviate ER stress, on the inhibition of P53 activation and apoptosis was investigated. As a result, it was confirmed that p53 activation and apoptosis caused by ER stress induced by combined treatment with cabozantinib and CBD were suppressed when LAA was treated (FIG. 11).

### Experimental Example 5: Analysis of Association Between Combined Treatment with Cannabidiol and Cabozantinib and CNR Receptor

CBD is known to bind to cannabinoid receptor 1 (CNR1) and CNR2 and regulate their downstream molecular pathways. Therefore, to determine whether the mechanism associated with combined treatment with cabozantinib and CBD is related to CNR1 and CNR2, the following experiments were conducted.

First, a CNR overexpression system was established in HepG2 cells, which show low endogenous expression levels of CNR1 and CNR2 (FIG. 12A). Next, the relationship between the combined treatment of cabozantinib and CBD and CNR1 and CNR2 was confirmed using the system, and it was confirmed that cell viability by the combined treatment was not related to CNR1 or CNR2 (FIG. 12B and Table 5).

TABLE 5

IC₅₀ (μM)

| Drug | without CBD | with CBD |
|---|---|---|
| pCDH-Ctrl | >100 | 16.02 |
| pCDH-CNR1 | >100 | 20.18 |
| pCDH-CNR2 | >100 | 18.24 |

Next, FACS analysis was conducted to evaluate whether the expression of CNR1 and CNR2 may modulate the apoptotic effect of combined treatment with cabozantinib and CBD. As a result, when cabozantinib and CBD were combine treated, cells expressing CNR1 and/or CNR2 exhibited similar levels of apoptosis, including early and late apoptotic cells, compared to control group cells (FIG. 13A).

Next, Western blotting analysis was conducted to evaluate whether the expression of CNR1 and CNR2 may affect the expression levels of apoptosis-related proteins following combined treatment with cabozantinib and CBD. As a result, when cabozantinib and CBD were combine treated, cells expressing CNR1 and/or CNR2 were found to have similar levels of apoptosis-related proteins compared to control group cells (FIG. 13B).

Summarizing the results, it may be seen that the cancer cell killing efficacy of combined treatment with cabozantinib and CBD does not depend on CNR1 and/or CNR2.

The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the examples described above should be understood in all respects as illustrative and not restrictive.

1. A method of treating or preventing liver cancer, which includes administering to a subject cannabidiol (CBD) and an anticancer agent.

2. The method of claim 1, wherein the cannabidiol and the anticancer agent are co-administered.

3. The method of claim 1, wherein the cannabidiol is administered simultaneously with the anticancer agent in a single formulation, or administered simultaneously or sequentially with the anticancer agent in a separate formulation.

4. The method of claim 1, wherein the cannabidiol and the anticancer agent are administered in a concentration ratio of 4:1 to 1:4.

5. The method of claim 1, wherein the anticancer agent comprises one or more selected from the group consisting of cabozantinib, sorafenib, lenvatinib, and regorafenib.

6. The method of claim 1, wherein the cannabidiol comprises one or more selected from the group consisting of derivatives of cannabidiol, stereoisomers thereof, pharmaceutically acceptable salts thereof, hydrates thereof, and solvates thereof.

7. The method of claim 1, wherein the method induces or enhances one or more of effects of apoptosis and autophagy of liver cancer cells.

8. A method of enhancing treating or the anticancer effect of liver cancer, which comprises administering to a subject a cannabidiol (CBD).

9. The method of claim 1, wherein the cannabidiol is administered in combination with an anticancer agent.

10. A pharmaceutical composition for preventing or treating liver cancer, comprising cannabidiol (CBD) and an anticancer agent as active ingredients.
